(19) United States
(12) Patent Application Publication
(10) Pub. No.: US 2022/0315600 A1
(43) Pub. Date: Oct. 6, 2022

(54) CANNABICITRAN COMPOSITIONS AND METHODS OF SYNTHESIZING CANNABICITRAN

(71) Applicant: Canopy Growth Corporation, Smiths Falls (CA)

(72) Inventors: Daniel BRUMAR, Smiths Falls (CA); Mahmood Azizpour FARD, Smiths Falls (CA); Ben GEILING, Smiths Falls (CA); Mohammadmehdi HAGHDOOST MANUJILI, Smiths Falls (CA)

(21) Appl. No.: 17/639,303

(22) PCT Filed: Dec. 24, 2020
(86) PCT No.: PCT/CA2020/051800

§ 371 Date: Feb. 28, 2022

Related U.S. Application Data
(60) Provisional application No. 63/009,269, filed on Apr. 13, 2020.
(60) Provisional application No. 62/953,374, filed on Dec. 24, 2019.

(51) Int. Cl. C07D 493/18

(52) U.S. Cl. CPC C07D 493/18

(57) ABSTRACT

Compositions having enhanced cannabicitran concentrations and/or enhanced cannabicitran-derivative concentrations are disclosed herein as are methods of synthesizing cannabicitran and/or cannabicitran derivatives. Relative to conventional methods, the methods of the present disclosure may: (i) be better suited to large-scale conditions in that they do not require dangerous and/or toxic solvents and/or reagents; (ii) be more tolerant of complex starting compositions, such as cannabinoid isolates and/or distillates; (iii) provide cannabicitran and/or cannabicitran derivatives at higher yield; (iv) provide easier to purify product mixtures comprising cannabicitran and/or cannabicitran derivatives; (v) provide product mixtures comprising cannabicitran or cannabicitran derivatives that are easier to purify; (vi) provide product mixtures that comprise unique ratios of cannabicitran or cannabicitran derivatives relative to other cannabinoids; and/or (vii) provide product mixtures with reduced THC concentrations.

### CROSS-REFERENCE TO RELATED APPLICATIONS

This application claims priority to and benefit of U.S. Provisional Patent Application Ser. No. 62/953,374 filed on Dec. 24, 2019 and U.S. Provisional Patent Application Ser. No. 63/009,269 filed on Apr. 13, 2020, each of which is hereby incorporated by reference in its entirety.

### TECHNICAL FIELD

The present disclosure generally relates to methods for synthesizing cannabinoids and to cannabinoid compositions having unique ratios of cannabinoids. In particular, the present disclosure relates to methods for synthesizing cannabicitran and/or a derivative thereof and to cannabinoid compositions having enhanced cannabicitran concentrations and/or enhanced cannabicitran-derivative concentrations.

### BACKGROUND

Cannabicitran is one of approximately ten main cannabinoids found in the Cannabis sativa plant, and it is quickly gaining consumer and scientific interest for both medical and recreational applications. For example, scientific studies indicate that cannabicitran has the potential to relieve intraocular pressure, and also to provide additional benefits in association with other cannabinoids via the entourage effect. Moreover, cannabicitran has potential utility as a synthon for chemical applications. However, since cannabicitran is typically found in low concentrations in Cannabis and/or Cannabis extracts, it is challenging to isolate. Accordingly, methods for synthesizing cannabicitran are attractive as are cannabinoid compositions that have enhanced cannabicitran concentrations.

Conventional methods for producing cannabicitran typically lack selectivity and lead to significant quantities of other products, for example cannabichromene (CBC) and/or tetrahydrocannabinol (THC). Isolating cannabicitran from reaction mixtures can pose significant challenges, as cannabicitran is difficult to separate from other products by typical purification methods (e.g. chromatography). This can make cannabicitran difficult to obtain in pure form. THC is psychoactive, and compositions containing THC may be subject to regulatory restrictions or prohibitions on transportation or sale. This has the potential to limit the utility of the cannabicitran compositions, because of the difficulty associated with separating the two cannabinoids. At the same time, conventional methods for synthesizing cannabicitran typically rely on toxic solvents and reagents, which have the potential to limit the utility of the resulting products. Moreover, conventional methods for producing cannabicitran are typically limited to small scale. Accordingly, improved methods for producing cannabicitran are desirable. At the same time, available cannabinoid compositions have low cannabicitran content—both in terms of absolute cannabicitran concentration and relative to other cannabinoids. Accordingly, compositions with enhanced cannabicitran concentration are also desirable.

### SUMMARY

The present disclosure is based on extensive research and development directed at overcoming at least some of the current impediments to advancing the start of the art in cannabicitran-related applications. As exemplified by the examples set out herein, the present disclosure advances this field with the provision of improved methods of synthesizing cannabicitran and/or a derivative thereof. The present disclosure also provides cannabinoid compositions having unique ratios of cannabinoids. The methods of the present disclosure may be better suited to industrial scale in that they do not require dangerous and/or toxic solvents and/or reagents, and in particular do not require the presence of a solvent when cannabicitran and/or a derivative thereof is formed. The methods of the present disclosure may provide for more efficient purification of cannabicitran and/or a derivative thereof. The methods of the present disclosure provides access to: (i) mixtures of cannabinoids, including mixtures of cannabichromene (CBC) and cannabicitran and/or derivatives thereof; (ii) high purity cannabicitran and/or derivatives thereof; and/or (iii) mixtures of cannabinoids with reduced THC concentrations. Importantly, the present disclosure also provides synthetic methods that are tolerant to a variety of complex starting compositions, such as Cannabis extracts and/or distillates.

In select embodiments, the present disclosure relates to a method of converting CBC or a CBC derivative to cannabicitran or a cannabicitran derivative, the method comprising heating the CBC or the CBC derivative to a reaction temperature of at least 60° C. for a reaction time of at least about 30 minutes to convert at least a portion of the CBC or the CBC derivative to cannabicitran or the cannabicitran derivative.

In select embodiments, the present disclosure relates to a method of preparing cannabicitran or a cannabicitran derivative, the method comprising: heating a reaction mixture comprising citral, a modified resorcinol, and an amine under a first set of reaction conditions to form a first product mixture; and heating at least a portion of the first product mixture under a second set of reaction conditions comprising a second reaction temperature of least 60° C., a second reaction pressure, and a second reaction time of at least about 30 minutes to form a second product mixture that has a cannabinoid content of which at least 10% w/w is cannabicitran or cannabicitran derivative.

In select embodiments, the present disclosure relates to a method of preparing a cannabinoid composition with enhanced cannabicitran concentration or enhanced cannabicitran-derivative concentration, the method comprising heating a first cannabinoid composition comprising CBC or a CBC derivative at a target reaction temperature, for a target reaction time, under a target reaction pressure to produce the cannabinoid composition with enhanced cannabicitran concentration or enhanced cannabicitran-derivative concentration at a target cannabicitran:CBC ratio that is greater than about 1:20 or at a target cannabicitran derivative:CBC derivative ratio that is greater than about 1:20.

In select embodiments, the present disclosure relates to a cannabinoid composition comprising at least about 1% w/w CBC or CBC derivative and at least about 1% w/w cannabicitran or cannabicitran derivative. In select embodiments of the present disclosure, the cannabinoid composition comprises between about 1% w/w CBC or CBC derivative and about 5% w/w CBC or CBC derivative, between about 5% w/w CBC or CBC derivative and about 25% w/w CBC or CBC derivative, or greater than about 25% w/w CBC or CBC derivative. In select embodiments of the present disclosure, the cannabinoid composition comprises between about 1% w/w cannabicitran or cannabicitran derivative and about 99% w/w cannabicitran or cannabicitran derivative, between about 10% w/w cannabicitran or cannabicitran derivative and about 90% w/w cannabicitran or cannabicitran derivative, between about 25% w/w cannabicitran or cannabicitran derivative and about 75% w/w cannabicitran or cannabicitran derivative, or between about 40% w/w cannabicitran or cannabicitran derivative and about 50% cannabicitran or cannabicitran derivative.

Other aspects and features of the present disclosure will become apparent to those ordinarily skilled in the art upon review of the following description of specific embodiments.

### BRIEF DESCRIPTION OF THE DRAWINGS

FIG. 1 shows high-performance liquid chromatograms for EXAMPLE 1 under initial conditions, and after 3, 6, and 24 hours.

FIG. 2 shows a high-performance liquid chromatogram for EXAMPLE 2 after purification by distillation.

FIG. 3 shows a high-performance liquid chromatogram for EXAMPLE 3 under initial conditions, and after 16, 40, and 90 hours.

FIG. 4 shows a plot of the changing cannabinoid profile over time for the reaction mixture of EXAMPLE 3.

### DETAILED DESCRIPTION

As noted above, the present disclosure provides improved methods of synthesizing cannabicitran and/or cannabicitran derivatives. Relative to conventional methods, the methods of the present disclosure may: (i) be better suited to large-scale conditions in that they do not require dangerous and/or toxic solvents and/or reagents; (ii) be more tolerant of complex starting compositions, such as cannabinoid isolates and/or distillates; (iii) provide cannabicitran and/or a derivative thereof at higher yield; (iv) provide easier to purify product mixtures comprising cannabicitran and/or a derivative thereof; (v) provide product mixtures that comprise unique ratios of cannabicitran and/or a derivative thereof relative to other cannabinoids; and/or (vi) provide product mixtures with reduced THC concentrations. While there may be little information available in the current research literature on pharma-kinetic interactions between cannabicitran and/or a derivative thereof and other cannabinoids, the present disclosure asserts that access to cannabicitran and/or a derivative thereof via the methods disclosed herein may be desirable in both medicinal and recreational contexts. Moreover, the present disclosure asserts that access to cannabicitran and/or a derivative thereof via the methods disclosure herein may be desirable to synthetic chemists.

The present disclosure provides cannabinoid compositions having unique ratios of cannabinoids, including unique ratios of cannabichromene (CBC) or a derivative thereof to cannabicitran or a derivative thereof. Mixtures of cannabinoids may provide enhanced medicinal and/or recreational effects, for example via the entourage effect. The present disclosure provides cannabinoid compositions having reduced THC concentrations which may thereby avoid being subject to regulatory restrictions or prohibitions on transportation or sale. In some embodiments, the methods of the present disclosure provide access to high purity cannabicitran and/or a derivative thereof, for example by converting CBC and/or a derivative thereof to primarily cannabicitran and/or a derivative thereof. High purity cannabicitran and/or a derivative thereof may be employed as an active pharmaceutical ingredient (API) for recreational and/or medicinal formulations, or as a synthon for chemical applications. High purity cannabicitran and/or a derivative thereof may be essentially free of THC and may be obtained from cannabinoid compositions having reduced THC concentrations. In some embodiments, the methods of the present disclosure may employ a Cannabis extract or a synthesis reaction mixture as a starting material, as select methods of the present disclosure may be compatible with impure starting materials.

Without being bound to any particular theory, the present disclosure asserts that converting CBC and/or a derivative thereof into cannabicitran and/or a derivative thereof as demonstrated herein results from an enthalpy-induced intramolecular cyclization reaction. The enthalpy-induced intramolecular cyclization of CBC and/or a derivative thereof may be performed in the absence of catalysts, for example acidic and/or free radical catalysts, and/or in the absence of UV light.

In select embodiments, the present disclosure relates to a method of converting CBC or a CBC derivative to cannabicitran or a cannabicitran derivative, the method comprising heating the CBC or the CBC derivative to a reaction temperature of at least 60° C. for a reaction time of at least about 30 minutes to convert at least a portion of the CBC or the CBC derivative to cannabicitran or the cannabicitran derivative.

In the context of the present disclosure, the terms “convert” and “converting” include a reaction involving a reagent initially present as a purified compound, an isolate, a component of a mixture (including a Cannabis extract or distillate), and/or a reagent formed in situ.

In the context of the present disclosure, the term “CBC” refers to cannabichromene or, more generally, cannabichromene-type cannabinoids. Accordingly the term “CBC” includes: (i) acid forms, such as “A-type”, “B-type”, or “AB-type” acid forms; (ii) salts of such acid forms, such as Na⁺ or Ca²⁺ salts of such acid forms; (iii) ester forms, such as those formed by hydroxyl-group esterification to form traditional esters, sulphonate esters, and/or phosphate esters; and/or (iv) various stereoisomers. CBC may have the following structural formula:

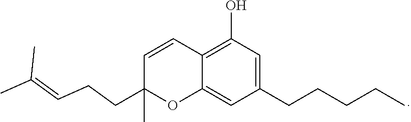 [SMILES: CCCCCc1cc(O)c2c(c1)OC(C)(CCC=C(C)C)C=C2]

In the context of the present disclosure, the term “cannabicitran” comprises cannabicitran-type cannabinoids. Accordingly the term “cannabicitran” includes: (i) acid forms, such as “A-type”, “B-type”, or “AB-type” acid forms; (ii) salts of such acid forms, such as Na⁺ or Ca²⁺ salts of such acid forms; (iii) ester forms, such as those formed by hydroxyl-group esterification to form traditional esters, sulphonate esters, and/or phosphate esters; and/or (iv) various stereoisomers. Cannabicitran may have the following structural formula:

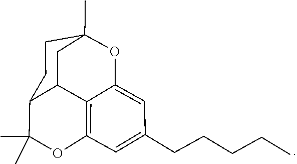 [SMILES: CCCCCc1cc2c3c(c1)OC(C)(C)C1CCC(C)(CC31)O2]

In select embodiments of the present disclosure, at least a portion of the heating is at a pressure of about 1 bar. In select embodiments of the present disclosure, at least a portion of the heating is at a pressure above about 1 bar. In select embodiments of the present disclosure, at least a portion of the heating is at a pressure below 1 bar, between about 0.1 mbar and 100 mbar, or between about 0.5 mbar and 10 mbar. In select embodiments of the present disclosure, at least a portion of the heating is at about 0.1 mbar, about 0.5 mbar, about 1 mbar, about 5 mbar, about 10 mbar, about 20 mbar, about 30 mbar, about 40 mbar, about 60 mbar, about 80 mbar, or about 100 mbar. Executing at least a portion of the heating at a reduced pressure may reduce the amount of side products present in a product of the reaction. Non-exclusive examples of side products include CBC, tetrahydrocannabinol (THC), cannabicyclol (CBL), cannabigerol (CBG), cannabidiol (CBD), and cannabinol (CBN).

In select embodiments of the present disclosure, the reaction temperature is between about 100° C. and about 150° C., between about 120° C. and about 130° C., or about 125° C. In select embodiments of the present disclosure, the reaction temperature is about 110° C., about 115° C., about 120° C., about 125° C., about 130° C., about 135° C., about 140° C., about 145° C., or about 150° C. In select embodiments of the present disclosure, the reaction temperature is between about 150° C. and about 220° C., or about 190° C. and about 210° C. In select embodiments of the present disclosure, the reaction temperature is about 150° C., about 155° C., about 160° C., about 165° C., about 170° C., about 175° C., about 180° C., about 185° C., about 190° C., about 195° C., about 200° C., about 205° C., or about 210° C. A higher temperature may increase the rate of reaction, and a higher rate of reaction may increase throughput and may therefore be more compatible with large scale reactions. A lower temperature may increase the yield of the reaction and/or reduce the amount of side products formed.

In select embodiments of the present disclosure, the reaction time is between 10 minutes and 100 hours. In select embodiments of the present disclosure, the reaction time is between about 10 minutes and about 48 hours, or between about 8 hours and about 24 hours. In select embodiments of the present disclosure, the reaction time is between about 10 minutes and about 1 hour, about 1 hour and about 8 hours, about 8 hours and about 24 hours, about 24 hours and about 100 hours, or greater than 100 hours. In select embodiments of the present disclosure, the reaction time is about 6 hours, about 12 hours, about 18 hours, about 24 hours, about 36 hours, about 48 hours, about 72 hours, about 96 hours, or greater than about 96 hours. A shorter reaction time may increase the throughput of the reaction, which may increase the rate at which cannabicitran or a derivative thereof is produced. A longer reaction time may increase the yield of the reaction.

In select embodiments of the present disclosure, a CBC derivative is a compound of the form:

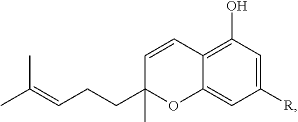

and a cannabicitran derivative is a compound of the form:

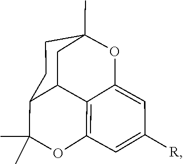

wherein R is methyl, ethyl, propyl, butyl, heptyl, 1,1-dimethylheptyl, phenylethyl, phenylvinyl, or benzofuran. As will be appreciated by those skilled in the art who have benefitted from the teachings of the present disclosure, a cannabicitran derivative converted from a CBC derivative will typically share the same R group. A cannabicitran derivative may possess enhanced or modified medicinal and/or recreational effect, and may provide additional functionality for synthetic chemistry and/or derivitization.

In select embodiments of the present disclosure, the CBC or a derivative thereof that is converted to cannabicitran or a derivative thereof is a component of a Cannabis composition, and the CBC or the derivative thereof accounts for at least about 50% w/w of the Cannabis composition. In select embodiments of the present disclosure, the CBC or the derivative thereof accounts for between about 50% and about 100%, about 50% and about 90%, about 50% and about 80%, about 50% and about 70%, or about 50% and about 60% w/w of the Cannabis composition. In select embodiments of the present disclosure, the CBC or the derivative thereof accounts for about 50%, about 60%, about 70%, about 80%, or about 90% w/w of the Cannabis composition. In select embodiments of the present disclosure, the Cannabis composition comprises at least 90% CBC or a derivative thereof. In the context of the present disclosure, a Cannabis composition may be a Cannabis extract. The methods of the present disclosure allow conversion of CBC or a derivative thereof to cannabicitran or a derivative thereof where the CBC or the derivative thereof is a component of a mixture (e.g. a Cannabis composition), which allows the use of reagents with reduced purity that may be more readily available or less expensive than reagents of higher purity.

In select embodiments of the present disclosure, the heating of the CBC or a derivative thereof produces a product mixture, and the method further includes purifying the product mixture to provide cannabicitran or a derivative thereof with a purity of at least 90% at a yield of at least 30%. In select embodiments of the present disclosure, purifying the product mixture may provide a purity of at least about 92%, at least about 94%, at least about 96%, at least about 98%, at least about 99%, or at least about 99.5%. In select embodiments of the present disclosure, purifying the product mixture may provide a yield of at least about 40%, at least about 50%, at least about 60%, at least about 70%, at least about 80%, or at least about 90%. High-purity cannabicitran and/or high-purity derivatives thereof may be more readily used as an API. Non-exclusive examples of purification methods include chromatography, flash chromatography, reversed phase C18 flash chromatography, simulated moving bed chromatography, liquid-liquid extraction, distillation, and short-path distillation. Chromatography methods may be effective for the isolation of cannabicitran and/or a derivative thereof from CBC and/or a derivative thereof.

In select embodiments of the present disclosure, the cannabicitran is a cannabicitran isomer.

In select embodiments of the present disclosure, the product mixture comprises less than about 30% w/w tetrahydrocannabinol (THC), less than about 25% w/w THC, less than about 20% w/w THC, less than about 15% w/w THC, less than about 10% w/w THC, less than about 5% w/w THC, less than about 1% w/w THC, less than about 0.3% w/w THC, less than about 0.2% w/w THC, or less than about 0.1% w/w THC. In the context of the present disclosure, the phrase “less than” includes amounts below the detection limit of appropriate analytical methods. A lower amount of THC may allow a product mixture to more easily satisfy regulatory restrictions.

In select embodiments of the present disclosure, a cannabicitran:THC ratio of the product mixture may be between about 1000:1 and about 1:1 or between about 100:1 and about 10:1. In select embodiments of the present disclosure, the cannabicitran:THC ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, or about 25:1 and about 10:1. As will be appreciated from the disclosure herein, reference to cannabicitran in this paragraph equally applies to cannabicitran derivatives.

In select embodiments of the present disclosure, the product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a cannabicitran:CBD ratio of the product mixture may be between about 100:1 and about 1:100, more particularly between about 100:1 and about 1:10. In select embodiments of the present disclosure, the cannabicitran:CBD ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the cannabicitran:CBD ratio of the product mixture may be about 10:1, about 5:1, about 2:1, about 1:1, about 1:2, about 1:5 or about 1:10. As will be appreciated from the disclosure herein, reference to cannabicitran in this paragraph equally applies to cannabicitran derivatives.

In select embodiments, the present disclosure relates to a method of preparing cannabicitran or a cannabicitran derivative, the method comprising: heating a reaction mixture comprising citral, a modified resorcinol, and an amine under a first set of reaction conditions comprising a first reaction temperature, a first reaction time, and a first reaction pressure to form a first product mixture; and heating at least a portion of the first product mixture under a second set of reaction conditions comprising a second reaction temperature of least 120° C., a second reaction time of at least about 30 minutes, and a second reaction pressure to form a second product mixture that has a cannabinoid content of which at least 10% w/w is cannabicitran or cannabicitran derivative.

In select embodiments of the present disclosure, the second reaction pressure is about 1 bar. In select embodiments of the present disclosure, the second reaction pressure is above about 1 bar. In select embodiments of the present disclosure, the second reaction pressure is below about 1 bar, between about 0.1 mbar and 100 mbar, and/or between about 0.5 mbar and 10 mbar. In select embodiments of the present disclosure, the second reaction pressure is at about 0.1 mbar, about 0.5 mbar, about 1 mbar, about 5 mbar, about 10 mbar, about 20 mbar, about 30 mbar, about 40 mbar, about 60 mbar, about 80 mbar, or about 100 mbar.

In select embodiments of the present disclosure, the second reaction temperature is between about 100° C. and about 150° C., between about 120° C. and about 130° C., or about 125° C. In select embodiments of the present disclosure, the second reaction temperature is about 110° C., about 115° C., about 120° C., about 125° C., about 130° C., about 135° C., about 140° C., about 145° C., or about 150° C. In select embodiments of the present disclosure, the second reaction temperature is between about 150° C. and about 220° C., or about 190° C. and about 210° C. In select embodiments of the present disclosure, the second reaction temperature is at about 150° C., about 155° C., about 160° C., about 165° C., about 170° C., about 175° C., about 180° C., about 185° C., about 190° C., about 195° C., about 200° C., about 205° C., or about 210° C. Increasing the second reaction temperature may increase the rate of reaction, while a lower temperature may increase the yield of the reaction or reduce the amount of side products formed.

In select embodiments of the present disclosure, the second reaction time is between 10 minutes and 100 hours. In select embodiments of the present disclosure, the second reaction time is between about 10 minutes and about 48 hours, or about 8 hours and about 24 hours. In select embodiments of the present disclosure, the second reaction time is between about 10 minutes and about 1 hour, about 1 hour and about 8 hours, about 8 hours and about 24 hours, about 24 hours and about 100 hours, or greater than 100 hours. In select embodiments of the present disclosure, the second reaction time is about 6 hours, about 12 hours, about 18 hours, about 24 hours, about 36 hours, about 48 hours, about 72 hours, about 96 hours, or greater than about 96 hours.

In the context of the present disclosure, the term “resorcinol” refers to resorcinol or, more generally, to resorcinol and modified resorcinols. Resorcinol is a compound of the following structure:

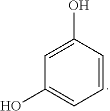

As used in the context of the present disclosure, and in relation to embodiments for the synthesis of cannabitran, the “resorcinol” includes the following as a modified resorcinol:

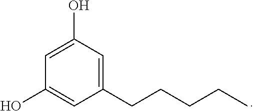

In select embodiments of the present disclosure, the modified resorcinol may be of the form:

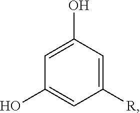

and the cannabicitran derivative may be of the form:

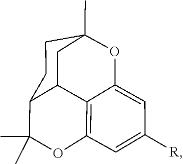

wherein R is methyl, ethyl, propyl, butyl, heptyl, 1,1-dimethylheptyl, phenylethyl, phenylvinyl, or benzofuran. As will be appreciated by those skilled in the art who have benefitted from the teachings of the present disclosure, a cannabicitran derivative prepared from a modified resorcinol will typically share the same R group.

In select embodiments of the present disclosure, the first product mixture comprises at least 90% w/w CBC or a derivative thereof. As discussed above, select methods of the present disclosure may be compatible with impure mixtures containing CBC or a derivative thereof below 100% w/w.

In select embodiments of the present disclosure, the method further comprises purifying the second product mixture to provide cannabicitran or a derivative thereof with a purity of at least 90% at a yield of at least 30%. In select embodiments of the present disclosure, purifying the second product mixture may provide a purity of at least about 92%, at least about 94%, at least about 96%, at least about 98%, at least about 99%, or at least about 99.5%. In select embodiments of the present disclosure, purifying the second product mixture may provide a yield of at least about 40%, at least about 50%, at least about 60%, at least about 70%, at least about 80%, or at least about 90%.

In select embodiments of the present disclosure, the amine comprises a primary amine, a secondary amine, a tertiary amine, or a combination thereof. For example the amine may comprise two amine groups: a primary amine and a secondary amine.

In select embodiments of the present disclosure, the first reaction conditions comprise contacting the citral, the modified resorcinol, and the amine with a class 3 solvent.

In select embodiments of the present disclosure, the first reaction temperature is between about 90° C. and about 115° C. In select embodiments, the first reaction temperature is about 90° C., about 95° C., about 100° C., about 105° C., about 110° C., about 115° C., or greater than 115° C. In select embodiments of the present disclosure, the first reaction time is less than about 30 minutes, about 30 minutes, about 1 hour, about 3 hours, about 8 hours, about 16 h, about 24 hours, or greater than about 24 hours. A lower first reaction temperature may increase the yield of the reaction, while a higher first reaction temperature may increase the rate of the reaction.

In select embodiments of the present disclosure, the first reaction pressure is below about 1 bar. In select embodiments of the present disclosure, the first reaction pressure is about 1 bar, between about 0.1 mbar and 100 mbar, and/or between about 0.5 mbar and 10 mbar. In select embodiments of the present disclosure, the first reaction pressure is at about 0.1 mbar, about 0.5 mbar, about 1 mbar, about 5 mbar, about 10 mbar, about 20 mbar, about 30 mbar, about 40 mbar, about 60 mbar, about 80 mbar, or about 100 mbar.

In select embodiments of the present disclosure, the second product mixture comprises between about 15% w/w and about 100% w/w cannabicitran or a derivative thereof, between about 50% w/w and 99% w/w cannabicitran or a derivative thereof, or between about 90% w/w and about 95% w/w cannabicitran or a derivative thereof. Control of the first reaction time, first reaction temperature, first reaction pressure, second reaction time, second reaction temperature, and second reaction pressure may allow control of the % w/w cannabicitran or cannabicitran derivative in the second product mixture.

In select embodiments of the present disclosure, the second product mixture comprises less than about 30% w/w tetrahydrocannabinol (THC), less than about 25% w/w THC, less than about 20% w/w THC, less than about 15% w/w THC, less than about 10% w/w THC, less than about 5% w/w THC, less than about 1% w/w THC, less than about 0.3% w/w THC, less than about 0.2% w/w THC, or less than about 0.1% w/w THC. In select embodiments of the present disclosure, a cannabicitran:THC ratio of the second product mixture is between about 1000:1 and about 1:1, or between about 100:1 and about 10:1. In select embodiments of the present disclosure, the cannabicitran:THC ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, or about 25:1 and about 10:1. As will be appreciated from the disclosure herein, reference to cannabicitran in this paragraph equally applies to cannabicitran derivatives.

In select embodiments of the present disclosure, the second product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a cannabicitran:CBD ratio of the second product mixture may be between about 100:1 and about 1:100, more particularly between about 100:1 and about 1:10. In select embodiments of the present disclosure, the cannabicitran:CBD ratio of the second product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the cannabicitran:CBD ratio of the second product mixture may be about 10:1, about 5:1, about 2:1, about 1:1, about 1:2, about 1:5 or about 1:10. As will be appreciated from the disclosure herein, reference to cannabicitran in this paragraph equally applies to cannabicitran derivatives.

In select embodiments, the present disclosure relates to a method of preparing a cannabinoid composition with enhanced cannabicitran concentration or enhanced cannabicitran-derivative concentration, the method comprising heating a cannabinoid composition comprising CBC or a CBC derivative at a target reaction temperature, for a target reaction time, under a target reaction pressure to produce the cannabinoid composition with enhanced cannabicitran concentration at a target cannabicitran:CBC ratio that is greater than about 1:20 or at a target cannabicitran derivative:CBC derivative ratio that is greater than about 1:20.

In the context of the present disclosure, the relative quantities of cannabicitran and CBC in a particular composition may be expressed as a ratio—cannabicitran:CBC. Likewise, in the context of the present disclosure, the relative quantities of a cannabicitran derivative and a CBC derivative in a particular composition may be expressed as a ratio—cannabicitran derivative:CBC derivative. Those skilled in the art will recognize that a variety of analytical methods may be used to determine such ratios, and the protocols required to implement any such method are within the purview of those skilled in the art. By way of non-limiting example, cannabicitran:CBC and/or cannabicitran derivative:CBC derivative ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. In select embodiments of the present disclosure, the compositions provided by the methods of the present disclosure have cannabicitran:CBC and/or cannabicitran derivative:CBC derivative ratios of greater than 1:20. In select embodiments of the present disclosure, the cannabicitran:CBC ratio or the cannabicitran derivative:CBC derivative ratio of the product mixture may be between about 1:20 and about 1000:1. In select embodiments of the present disclosure, the cannabicitran:CBC ratio or the cannabicitran derivative:CBC derivative ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:20.

In select embodiments of the present disclosure, the target reaction pressure is at about 1 bar. In select embodiments of the present disclosure, the target reaction pressure is above about 1 bar. In select embodiments of the present disclosure, the target reaction pressure comprises a pressure below about 1 bar, such as between about 0.1 mbar and 100 mbar, or between about 0.5 mbar and 10 mbar. In select embodiments of the present disclosure, the target reaction pressure is at about 0.1 mbar, about 0.5 mbar, about 1 mbar, about 5 mbar, about 10 mbar, about 20 mbar, about 30 mbar, about 40 mbar, about 60 mbar, about 80 mbar, or about 100 mbar.

In select embodiments of the present disclosure, the target reaction temperature is between about 100° C. and about 150° C., between about 120° C. and about 130° C., or about 125° C. In select embodiments of the present disclosure, the target reaction temperature is at about 110° C., about 115° C., about 120° C., about 125° C., about 130° C., about 135° C., about 140° C., about 145° C., or about 150° C. In select embodiments of the present disclosure, the target reaction temperature is between about 150° C. and about 220° C., or between about 190° C. and about 210° C. In select embodiments of the present disclosure, the target reaction temperature is about 150° C., about 155° C., about 160° C., about 165° C., about 170° C., about 175° C., about 180° C., about 185° C., about 190° C., about 195° C., about 200° C., about 205° C., or about 210° C.

In select embodiments of the present disclosure, the target reaction time is between 10 minutes and 100 hours. In select embodiments of the present disclosure, the target reaction time is between about 10 minutes and about 48 hours, or between about 8 hours and about 24 hours. In select embodiments of the present disclosure, the target reaction time is between about 10 minutes and about 1 hour, between about 1 hour and about 8 hours, between about 8 hours and about 24 hours, between about 24 hours and about 100 hours, or greater than 100 hours. In select embodiments of the present disclosure, the target reaction time is about 6 hours, about 12 hours, about 18 hours, about 24 hours, about 36 hours, about 48 hours, about 72 hours, about 96 hours, or greater than about 96 hours.

In select embodiments of the present disclosure, the first cannabinoid composition comprises a CBC derivative of the form:

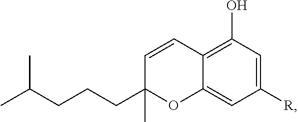

and the cannabicitran derivative is of the form:

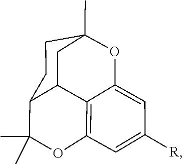

wherein R is methyl, ethyl, propyl, butyl, heptyl, 1,1-dimethylheptyl, phenylethyl, phenylvinyl, or benzofuran.

In select embodiments of the present disclosure, the first cannabinoid composition is a cannabinoid distillate, extract, or isolate. The Cannabis extract may be obtained from extraction of Cannabis plant material. The Cannabis distillate may be obtained from distillation of a mixture comprising cannabinoids, for example a Cannabis extract. The Cannabis isolate may be obtained via purification of a mixture comprising cannabinoids, for example crystallization of the Cannabis distillate or extract.

In select embodiments of the present disclosure, the method further comprises purifying the cannabinoid composition with enhanced cannabicitran concentration or enhanced cannabicitran-derivative concentration to provide cannabicitran or a cannabicitran derivative with a purity of at least 90% at a yield of at least 30%. In select embodiments of the present disclosure, purifying the cannabinoid composition with enhanced cannabicitran concentration or enhanced cannabicitran-derivative concentration may provide a purity of at least about 92%, at least about 94%, at least about 96%, at least about 98%, at least about 99%, or at least about 99.5%. In select embodiments of the present disclosure, purifying the product mixture may provide a yield of at least about 40%, at least about 50%, at least about 60%, at least about 70%, at least about 80%, or at least about 90%.

In select embodiments of the present disclosure, the target cannabicitran:CBC ratio or the target cannabicitran derivative:CBC derivative ratio is between about 1000:1 and about 1:20, about 100:1 and about 1:10, or about 95:1 and about 1:1. A higher target cannabicitran:CBC ratio or cannabicitran derivative:CBC derivative ratio provides a product with higher cannabicitran or cannabicitran derivative content. In select embodiments of the present disclosure, the first cannabinoid composition is at least 90% w/w CBC or CBC derivative. A higher % w/w CBC in the first cannabinoid composition may increase the yield of cannabicitran. A higher % w/w CBC derivative in the first cannabinoid composition may increase the yield of cannabicitran derivative.

In select embodiments of the present disclosure, the cannabinoid composition with enhanced cannabicitran concentration or the cannabinoid composition with enhanced cannabicitran-derivative concentration comprises less than about 30% w/w tetrahydrocannabinol (THC), less than about 10% w/w THC, less than about 1% w/w THC, less than about 0.3% w/w THC, or less than about 0.1% w/w THC. In select embodiments of the present disclosure, the cannabicitran:THC ratio is between about 1000:1 and about 1:1, or between about 100:1 and about 10:1. In select embodiments of the present disclosure, the cannabicitran derivative:THC ratio is between about 1000:1 and about 1:1 or between about 100:1 and about 10:1.

In select embodiments of the present disclosure, the cannabinoid composition comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a cannabicitran:CBD ratio of the cannabinoid composition may be between about 100:1 and about 1:100, more particularly between about 100:1 and about 1:10. In select embodiments of the present disclosure, the cannabicitran:CBD ratio of the cannabinoid composition may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the cannabicitran:CBD ratio of the cannabinoid composition may be about 10:1, about 5:1, about 2:1, about 1:1, about 1:2, about 1:5 or about 1:10. As will be appreciated from the disclosure herein, reference to cannabicitran in this paragraph equally applies to cannabicitran derivatives.

In select embodiments, the present disclosure relates to a cannabinoid composition comprising at least about 1% w/w CBC or CBD derivative and at least about 1% w/w cannabicitran or cannabicitran derivative.

In select embodiments of the present disclosure, a cannabicitran:CBC ratio of the cannabinoid composition is between about 1000:1 and about 1:20, between about 100:1 and about 1:10, or between about 95:1 and about 1:1. In select embodiments of the present disclosure, a cannabicitran derivative:CBC derivative ratio of the cannabinoid composition is between about 1000:1 and about 1:20, between about 100:1 and about 1:10, or between about 95:1 and about 1:1.

In select embodiments of the present disclosure, the CBC derivative is of the form:

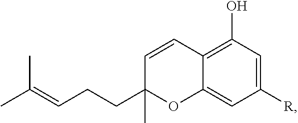

and

the cannabicitran derivative is of the form:

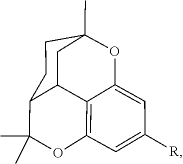

wherein R is methyl, ethyl, propyl, butyl, heptyl, 1,1-dimethylheptyl, phenylethyl, phenylvinyl, or benzofuran.

### Examples

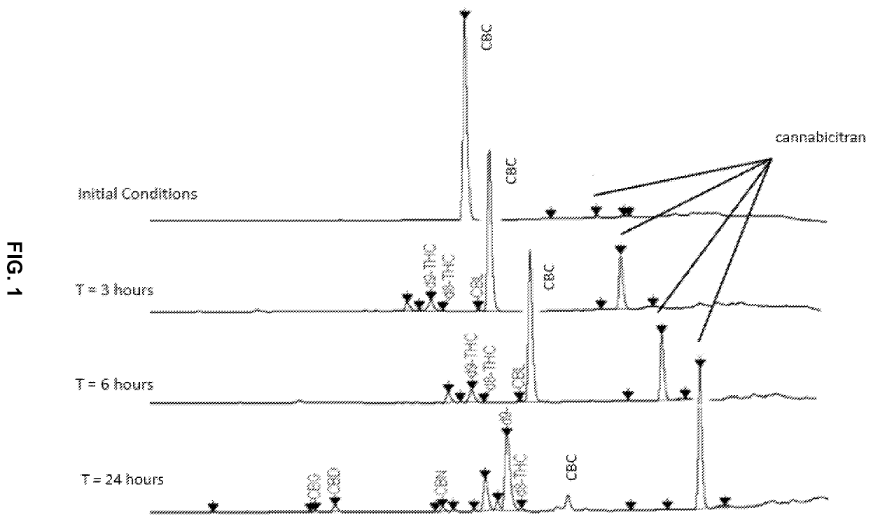

EXAMPLE 1: Cannabichromene (CBC) (5 g, prepared from citral and olivetol) was transferred to a pear-shaped flask equipped with a Schleck tube equipped with a thermometer and glass rod. The reaction was heated at 200° C. for 24 hours at pressure of 0.3 bar under solvent-less conditions to provide a crude cannabicitran resin. HPLC was also used to monitor reaction progress over time. HPLC chromatograms from reaction times of 0 h, 3 h, 6 h, and 24 h are shown in FIG. 1, and cannabinoid percentage values derived therefrom are set out in TABLE 1.

TABLE 1
HPLC results from EXAMPLE 1. Percentage
values for CBC and cannabicitran
were determined by HPLC-DAD (215 nm).

| Time (hours) | CBC (% w/w) | cannabicitran (% w/w) |
|---|---|---|
| 0 | >90 | trace |
| 3 | 83 | 12 |
| 6 | 76 | 14 |
| 24 | trace | 33 |

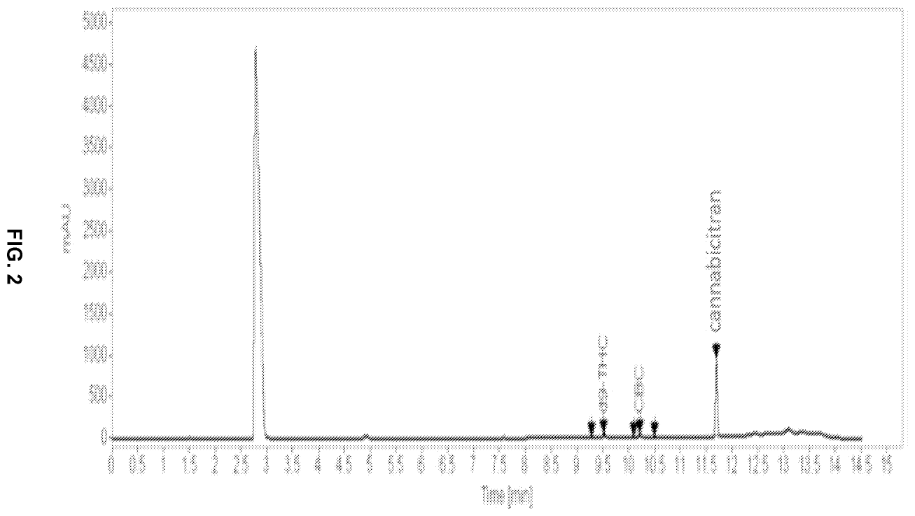

EXAMPLE 2: The crude cannabicitran resin of EXAMPLE 1 was purified by distillation at 100° C. and 0.34 mbar to give a purified cannabicitran resin with 96% w/w purity. An HPLC chromatogram for the purified cannabicitran resin is shown in FIG. 2.

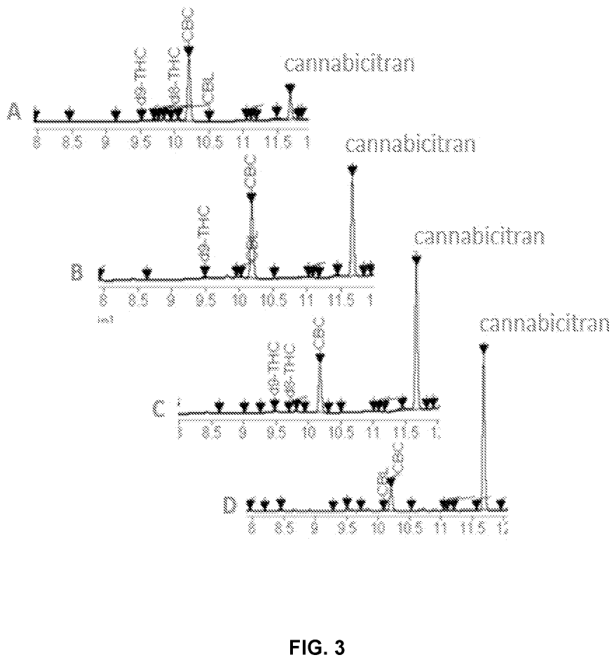

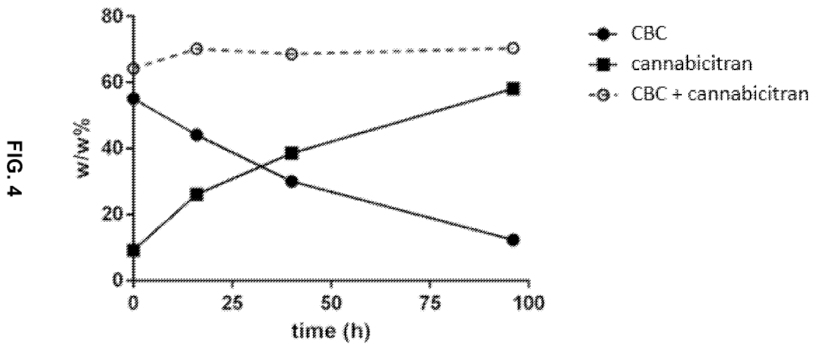

EXAMPLE 3: A synthetic cannabichromene (CBC) resin prepared in accordance with EXAMPLE 1 (3.320 g of resin comprising 55% w/w CBC and 9% w/w cannabicitran) was transferred into a test tube equipped with a stir bar. The reaction was heated at 125° C. while stirring at 200 rpm at atmospheric pressure for 96 hours. HPLC was also used to monitor reaction progress over time. HPLC chromatograms from reaction times of 0 h, 16 h, 40 h, and 90 h are shown in FIG. 3, and cannabinoid percentage values derived therefrom are set out in TABLE 2. Importantly, the HPLC analysis did not detect significant quantities of tetrahydrocannabinol (THC). FIG. 4 shows a plot of the changing cannabinoid profile of the reaction mixture over time.

TABLE 2
HPLC results from EXAMPLE 3. Percentage
values for CBC and cannabicitran
were determined by HPLC-DAD (215 nm).

| Time | CBC | cannabicitran | Reference |
|---|---|---|---|
| (hours) | (% w/w) | (% w/w) | in FIG. 3 |
| 0 | 55.07 | 9.17 | A |
| 16 | 44.07 | 26.13 | B |
| 40 | 30.02 | 38.59 | C |
| 96 | 16.80 | 49.41 | D |

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of the various components and steps”. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A method of converting cannabichromene (CBC) or a CBC derivative to cannabicitran or a cannabicitran derivative, the method comprising heating the CBC or the CBC derivative at a pressure below about 1 bar and in the absence of a solvent.

2.-5. (canceled)

6. The method of claim 1, wherein the heating is at a temperature between about 100° C. and about 150° C.

7. The method of claim 6, wherein the temperature is between about 120° C. and about 130° C.

8. The method of claim 1, wherein the heating is at a temperature between about 150° C. and about 220° C.

9. The method of claim 8, wherein the temperature is between about 190° C. and about 210° C.

10.-13. (canceled)

14. The method of claim 1, wherein the CBC derivative is of the form: 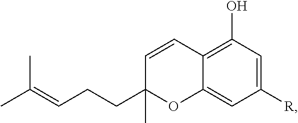
and
the cannabicitran derivative is of the form: 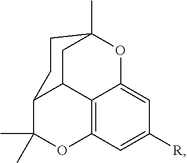
wherein R is methyl, ethyl, propyl, butyl, heptyl, 1,1-dimethylheptyl, phenylethyl, phenylvinyl, or benzofuran.

15.-25. (canceled)

26. A method of preparing cannabicitran or a cannabicitran derivative, the method comprising:
heating a reaction mixture comprising citral, a modified resorcinol, and an amine under a first set of reaction conditions to form a first product mixture; and
heating at least a portion of the first product mixture, under a second set of reaction conditions to form cannabicitran or cannabicitran derivative.

27. The method of claim 26, wherein the second set of reaction conditions comprises a pressure below about 1 bar.

28. (canceled)

29. The method of claim 26, wherein the second set of reaction conditions comprises a pressure at about 1 bar.

30. The method of claim 26, wherein the second set of reaction conditions is in the absence of a solvent.

31. The method of claim 26, wherein the second set of reaction conditions comprises a temperature between about 100° C. and about 150° C.

32. The method of claim 31, wherein the second temperature is between about 120° C. and about 130° C.

33. The method of claim 26, wherein the second set of reaction conditions comprises a temperature between about 150° C. and about 220° C.

34. (canceled)

35.-38. (canceled)

39. The method of claim 26, wherein the modified resorcinol is of the form:

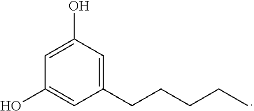

40. The method of claim 26, wherein the modified resorcinol is of the form: 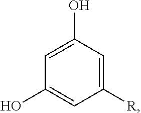
and
the cannabicitran derivative is of the form: 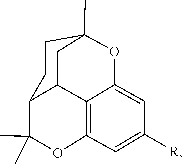
wherein R is methyl, ethyl, propyl, butyl, heptyl, 1,1-dimethylheptyl, phenylethyl, phenylvinyl, or benzofuran.

41.-44. (canceled)

45. The method of claim 26, wherein the amine comprises a primary amine, a secondary amine, a tertiary amine, or a combination thereof.

46. The method of claim 26, wherein the amine comprises a class 3 solvent, a natural amine, or a combination thereof.

47. The method of claim 26, wherein the first set of reaction conditions comprise contacting the citral, the modified resorcinol, and the amine with a class 3 solvent.

48.-94. (canceled)

95. A method of converting cannabichromene (CBC) or a CBC derivative to cannabicitran or a cannabicitran derivative, the method comprising heating the CBC or the CBC derivative at a temperature between 100° C. and 150° C., at atmospheric pressure, and for a time of at least 8 hours.

96. The method of claim 95, wherein the CBC derivative is of the form: 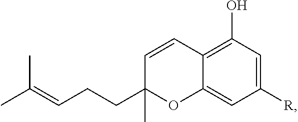
and
the cannabicitran derivative is of the form: 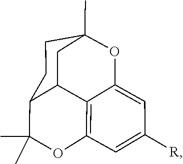
wherein R is methyl, ethyl, propyl, butyl, heptyl, 1,1-dimethylheptyl, phenylethyl, phenylvinyl, or benzofuran.
